(19) United States
(12) Patent Application Publication
(10) Pub. No.: US 2012/0330176 A1
(43) Pub. Date: Dec. 27, 2012

(54) NASAL CANNULA FOR CARBON DIOXIDE SAMPLING

(76) Inventor: Ngah-Chai Leow, Selangor (MY)

(21) Appl. No.: 13/580,853

(22) PCT Filed: Nov. 12, 2010
(86) PCT No.: PCT/MY2010/000275

§ 371 Date: Aug. 23, 2012

(30) Foreign Application Priority Data
Apr. 14, 2010 (MY) PI 2010001661

(51) Int. Cl. A61B 5/08

(52) U.S. Cl. 600/532

(57) ABSTRACT

A device to facilitate carbon dioxide sampling in the exhalation breath of a person comprising a single nasal prong (10) insertable into a nostril of the person having a first lumen (20) which allows gas passage from the nostril to the surrounding external air, a second lumen (34) located within the first lumen (20). A tube (40) is connected at a first end (42) to an end of the second lumen (34) that is further from the nostril and connectable at a second end (44) to a carbon dioxide monitoring device (50). The carbon dioxide monitoring device (50) detects the level of carbon dioxide in the portion of the person's exhalation that passes through the second lumen (34). The portion of the person's exhalation that passes through the first lumen (20) is dispersed in the surrounding air.

### FIELD OF INVENTION

The present invention relates generally to a device to facilitate carbon dioxide sampling in the exhalation and more specifically to such a device having a single nasal prong.

### BACKGROUND OF INVENTION

There are many devices that can measure the level of carbon dioxide in the exhalation of a person. For example, U.S. Pat. No. 1,926,027 describes a breathing apparatus which has a tubular connection with a relief port. U.S. Pat. No. 2,248,477 concerns an inhalator mask which has an inner chamber in gaseous communication with an outer chamber. U.S. Pat. No. 2,795,223 describes apparatus made up of a tube having a mouthpiece, the tube also having a by-pass duct communicating with a chamber. In U.S. Pat. No. 4,201,205 is disclosed use of non-elastic flexible tubing which extends over and around the user's ears and under his chin. U.S. Pat. No. 4,258,710 involves a respirator which has holes in the side of a mask and a cup in front with an opening including valve means having an outlet into the interior of the mask. In U.S. Pat. No. 4,328,797 is described a mask which is adapted to provide for the passage of a tube therethrough. U.S. Pat. No. 5,005,571 involves a mouth nose mask having breathing monitor or the like apparatus mounted on a patient's face independent of a nasal cannula and over the cannula without impeding its function. U.S. Pat. No. 5,046,491 deals with collection and transportation separately or simultaneously of gases inhaled or exhaled via a patient's mouth during anesthesia or the like, using a nasal gas cannula and an oral gas capture member. However, none of the foregoing art solves the problem of how to monitor the carbon dioxide level in the exhalation of a person using only a single nasal cannula.

There is therefore a need in the art for a device to facilitate carbon dioxide sampling in the exhalation of a person having a single nasal prong.

### SUMMARY OF INVENTION

This invention relates to a device to facilitate carbon dioxide sampling in the exhalation breath of a person comprising a single nasal prong insertable into a nostril of the person and allowing gas passage from the nostril into the nasal prong, and the prong having a first lumen, the first lumen allowing gas passage from the nostril to the surrounding external air. The nasal prong also having a second lumen, whereby second lumen is located within the first lumen. The first and second lumens are circular in cross section. An elongated hollow tube is connected at a first end to an end of the second lumen that is further from the nostril and connectable at a second end to a carbon dioxide monitoring device. The carbon dioxide monitoring device detects the level of carbon dioxide in the portion of the person's exhalation that passes through the second lumen. The portion of the person's exhalation that passes through the first lumen is dispersed in the surrounding air. The first lumen also allows inhalation of fresh air by the person.

In a first embodiment, the second lumen is located at an off center position within the first lumen such that an outer circumference of the second lumen is in contact with an inner circumference of the first lumen and the second lumen is the same length as the first lumen.

In a second embodiment, the second lumen is located at an off center position within the first lumen such that an outer circumference of the second lumen is in contact with an inner circumference of the first lumen and the second lumen is about half the length of the first lumen. In this embodiment, the second lumen starts from about half way down the first lumen and ends flush with an end of the first lumen that is further from the nostril.

In a third embodiment, the second lumen is located at an off center position within the first lumen such that an outer circumference of the second lumen is in contact with an inner circumference of the first lumen and the second lumen is about half the length of the first lumen. The second lumen starts from about half way down the first lumen and ends flush with an end of the first lumen that is further from the nostril. In this embodiment, there is a central wall running the length of the first lumen and dividing the first lumen into two halves, with the second lumen located within one of the two halves at an end further from the nostril.

In a fourth embodiment, the second lumen is located at a center position within the circumference of the first lumen so that there is an empty donut shaped space between the first and second lumens. The second lumen is supported by radial supports extending from the inner circumference of the first lumen to the outer circumference of the second lumen. In this embodiment, there are two such radial supports supporting the second lumen and evenly spaced around the circumference.

In a fifth embodiment, the second lumen is located at a center position within the circumference of the first lumen so that there is an empty donut shaped space between the first and second lumens. The second lumen is supported by radial supports extending from the inner circumference of the first lumen to the outer circumference of the second lumen. In this embodiment, there are three such radial supports supporting the second lumen and evenly spaced around the circumference.

In all embodiments, the nasal prong has an enlarged portion and is constructed of flexible material at the end that is inserted into the nostril to provide for easier insertion into nostril of various sizes.

There may also be different sized nasal prongs to cater for a wider range of nostril sizes.

### BRIEF DESCRIPTION OF DRAWINGS

FIGS. 1a, 1b and 1c show views of a device to facilitate carbon dioxide sampling in a first embodiment of this invention.

FIGS. 2a, 2b and 2c show views of a device to facilitate carbon dioxide sampling in a second embodiment of this invention.

FIGS. 3a and 3b show views of a device to facilitate carbon dioxide sampling in a third embodiment of this invention.

FIGS. 4a and 4b show views of a device to facilitate carbon dioxide sampling in a fourth embodiment of this invention.

FIGS. 5a and 5b show views of a device to facilitate carbon dioxide sampling in a fifth embodiment of this invention.

### DETAILED DESCRIPTION OF INVENTION

It should be noted that the following detailed description is directed to a device to facilitate carbon dioxide sampling in the exhalation breath of a person and is not limited to any particular size or configuration but in fact a multitude of sizes and configurations within the general scope of the following description.

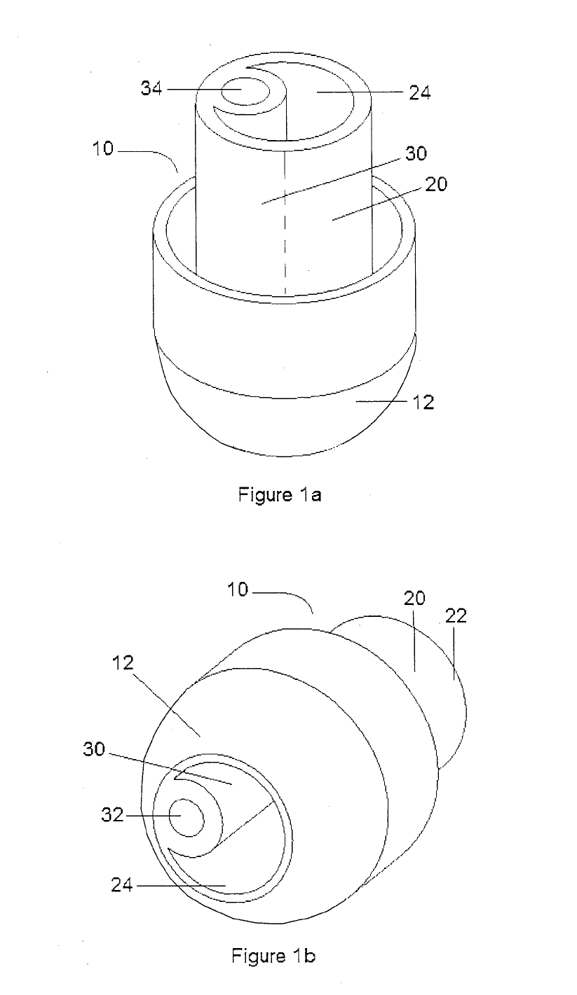

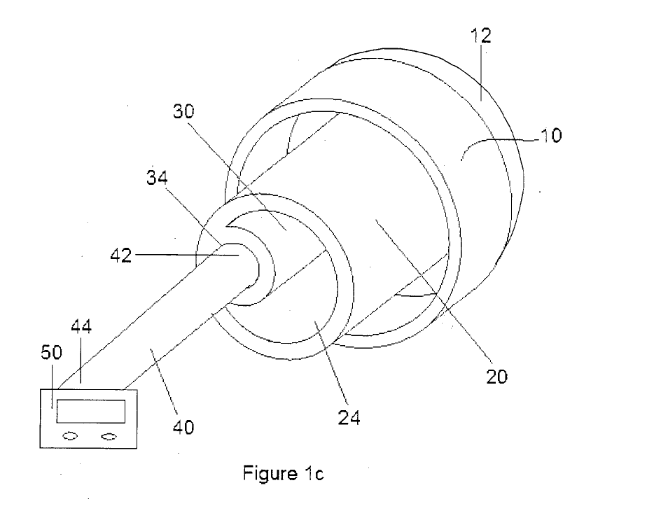

Referring to FIGS. 1a, 1b and 1c, there is shown a device to facilitate carbon dioxide sampling in the exhalation breath of a person in a first embodiment of this invention. A single nasal prong (10) insertable into a nostril of said person and allowing gas passage from the nostril into the nasal prong and having a first lumen (20), said first lumen allowing gas passage from the said nostril to the surrounding external air. The nasal prong (10) also having a second lumen (30), whereby said second lumen (30) is located within said first lumen (20). The first (20) and second (30) lumens are circular in cross section. An elongated hollow tube (40) is connected at a first end (42) to an end of said second lumen (34) that is further from the nostril and connectable at a second end (44) to a carbon dioxide monitoring device (50). The carbon dioxide monitoring device (50) detects the level of carbon dioxide in the portion of the person's exhalation that passes through the second lumen (30). The portion of the person's exhalation that passes through the first lumen (20) is dispersed in the surrounding air. The first lumen (20) also allows inhalation of fresh air by the person. In this embodiment, the second lumen (30) is located at an off center position within the said first lumen (20) such that an outer circumference of the second lumen is in contact with an inner circumference of the first lumen and the second lumen (30) is the same length as the first lumen (20). The nasal prong (10) has an enlarged portion and is constructed of flexible material at the end (12) that is inserted into the nostril to provide for easier insertion into nostril of various sizes.

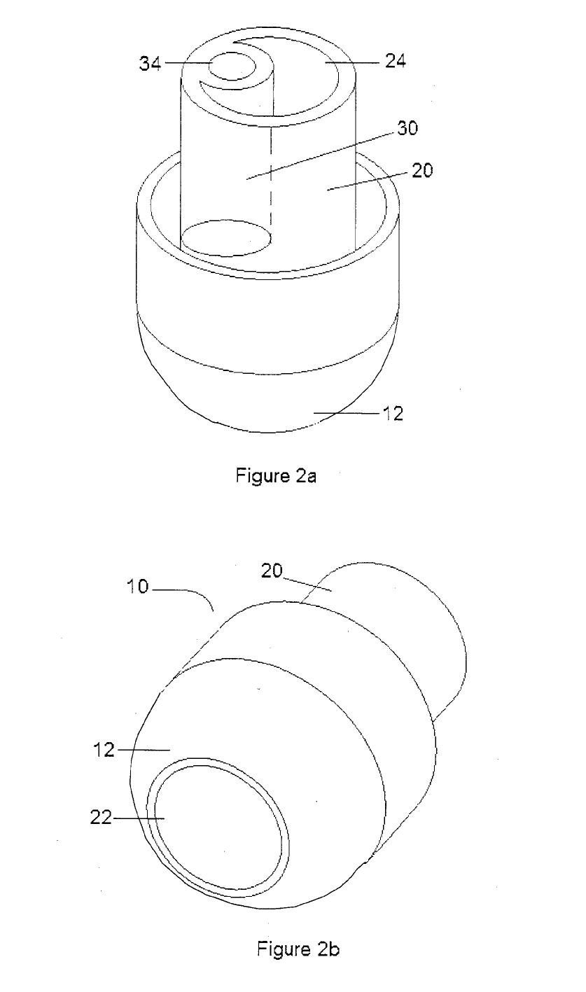

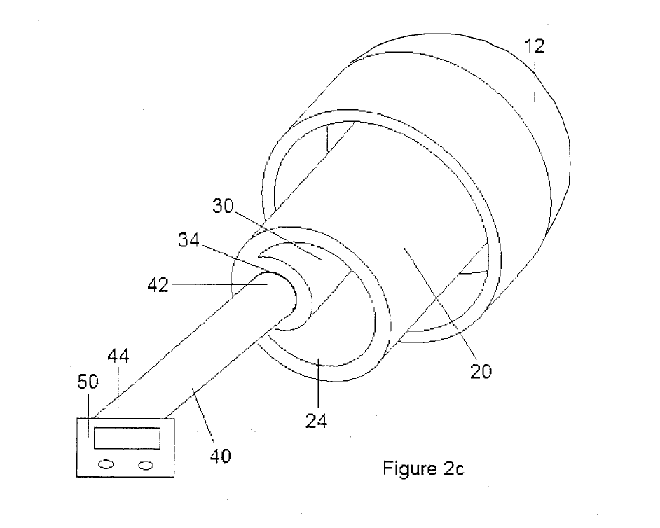

Referring to FIGS. 2a, 2b and 2c, there is shown a device to facilitate carbon dioxide sampling in the exhalation breath of a person in a second embodiment of this invention. A single nasal prong (10) insertable into a nostril of said person and allowing gas passage from the nostril into the nasal prong and having a first lumen (20), said first lumen allowing gas passage from the said nostril to the surrounding external air. The nasal prong (10) also having a second lumen (30), whereby said second lumen (30) is located within said first lumen (20). The first (20) and second (30) lumens are circular in cross section. An elongated hollow tube (40) is connected at a first end (42) to an end of said second lumen (34) that is further from the nostril and connectable at a second end (44) to a carbon dioxide monitoring device (50). The carbon dioxide monitoring device (50) detects the level of carbon dioxide in the portion of the person's exhalation that passes through the second lumen (30). The portion of the person's exhalation that passes through the first lumen (20) is dispersed in the surrounding air. The first lumen (20) also allows inhalation of fresh air by the person. In this embodiment, the second lumen (30) is located at an off center position within the said first lumen (20) such that an outer circumference of the second lumen is in contact with an inner circumference of the first lumen and the second lumen (30) is about half the length of the first lumen (20). In this embodiment, the second lumen (30) starts from about half way down the first lumen (20) and ends flush with an end of the first lumen (24) that is further from the nostril. The nasal prong (10) has an enlarged portion and is constructed of flexible material at the end (12) that is inserted into the nostril to provide for easier insertion into nostrils of various sizes.

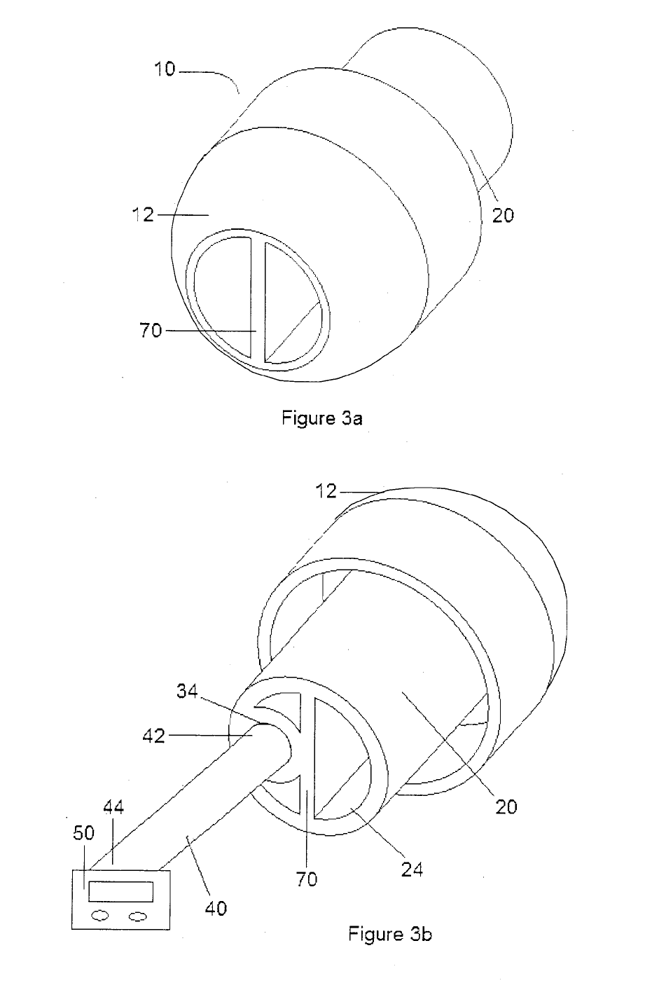

Referring to FIGS. 3a and 3b, there is shown a device to facilitate carbon dioxide sampling in the exhalation breath of a person in a third embodiment of this invention. A single nasal prong (10) insertable into a nostril of said person and allowing gas passage from the nostril into the nasal prong and having a first lumen (20), said first lumen allowing gas passage from the said nostril to the surrounding external air. The nasal prong (10) also having a second lumen (30), whereby said second lumen (30) is located within said first lumen (20). The first (20) and second (30) lumens are circular in cross section. An elongated hollow tube (40) is connected at a first end (42) to an end of said second lumen (34) that is further from the nostril and connectable at a second end (44) to a carbon dioxide monitoring device (50). The carbon dioxide monitoring device (50) detects the level of carbon dioxide in the portion of the person's exhalation that passes through the second lumen (30). The portion of the person's exhalation that passes through the first lumen (20) is dispersed in the surrounding air. The first lumen (20) also allows inhalation of fresh air by the person. In this embodiment, the second lumen (30) is located at an off center position within the said first lumen (20) such that an outer circumference of the second lumen is in contact with an inner circumference of the first lumen and the second lumen (30) is about half the length of the first lumen (20). The second lumen (30) starts from about half way down the first lumen (20) and ends flush with an end of the first lumen (24) that is further from the nostril. There is a central wall (70) running the length of the first lumen (20) and dividing the first lumen (20) into two halves, with the second lumen (30) located within one of said halves at an end (24) further from the nostril. The nasal prong (10) has an enlarged portion and is constructed of flexible material at the end (12) that is to be inserted into the nostril to provide for easier insertion into nostrils of various sizes.

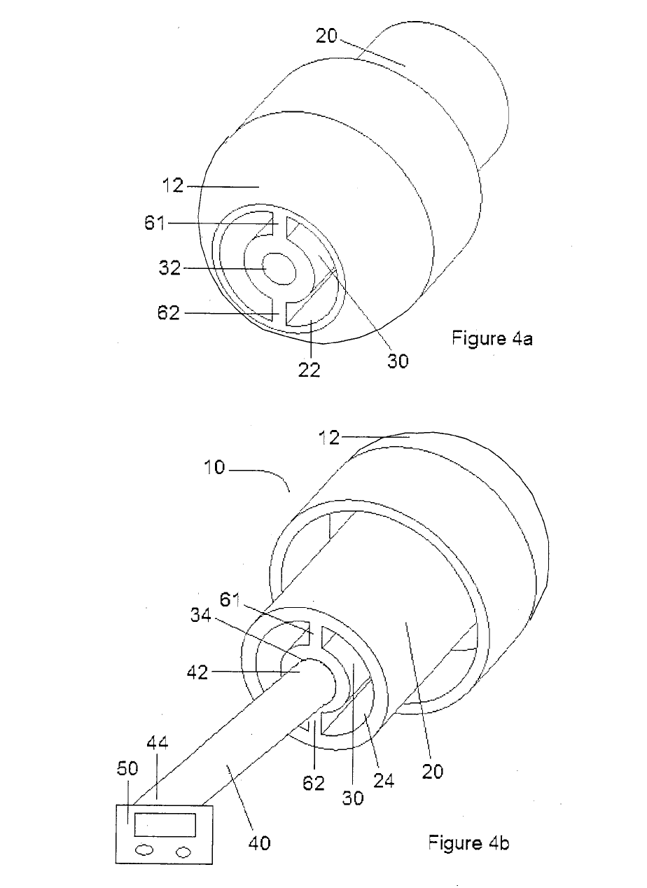

Referring to FIGS. 4a and 4b, there is shown a device to facilitate carbon dioxide sampling in the exhalation breath of a person in a fourth embodiment of this invention. A single nasal prong (10) insertable into a nostril of said person and allowing gas passage from the nostril into the nasal prong and having a first lumen (20), said first lumen allowing gas passage from the said nostril to the surrounding external air. The nasal prong (10) also having a second lumen (30), whereby said second lumen (30) is located within said first lumen (20). The first (20) and second (30) lumens are circular in cross section. An elongated hollow tube (40) is connected at a first end (42) to an end of said second lumen (34) that is further from the nostril and connectable at a second end (44) to a carbon dioxide monitoring device (50). The carbon dioxide monitoring device (50) detects the level of carbon dioxide in the portion of the person's exhalation that passes through the second lumen (30). The portion of the person's exhalation that passes through the first lumen (20) is dispersed in the surrounding air. The first lumen (20) also allows inhalation of fresh air by the person. In this embodiment, the second lumen (30) is located at a center position within the circumference of the first lumen (20) so that there is an empty donut shaped space between the first and second lumens. The second lumen (30) is supported by radial supports (61, 62) extending from the inner circumference of the first lumen to the outer circumference of the second lumen. In this embodiment, there are two such radial supports (61, 62) supporting the second lumen (30) and evenly spaced around the circumference. The nasal prong (10) has an enlarged portion and is constructed of flexible material at the end (12) that is inserted into the nostril to provide for easier insertion into nostril of various sizes.

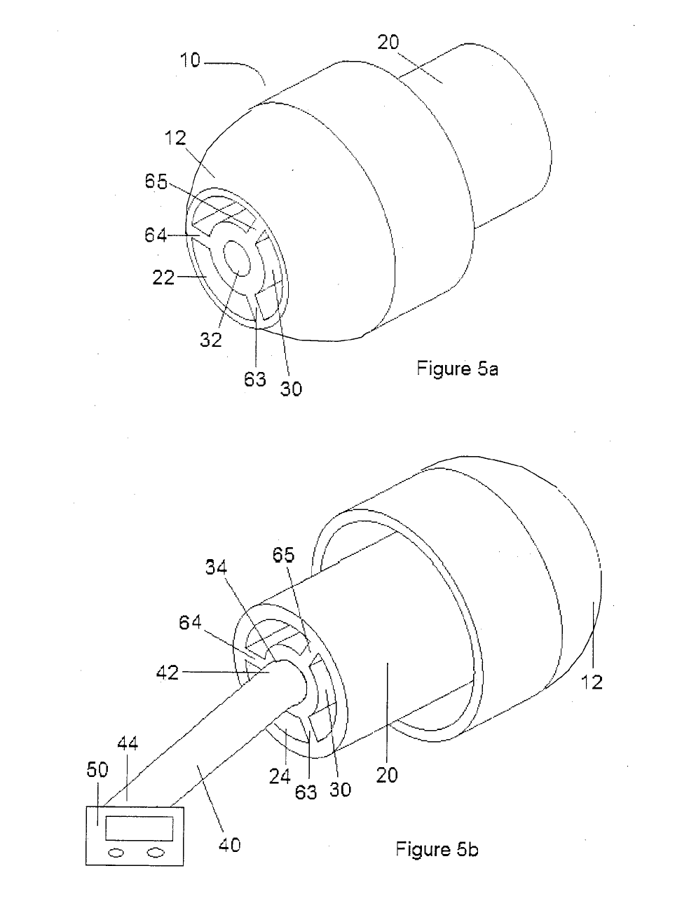

Referring to FIGS. 5a and 5b, there is shown a device to facilitate carbon dioxide sampling in the exhalation breath of a person in a fifth embodiment of this invention. A single nasal prong (10) insertable into a nostril of said person and allowing gas passage from the nostril into the nasal prong and having a first lumen (20), said first lumen allowing gas passage from the said nostril to the surrounding external air. The nasal prong (10) also having a second lumen (30), whereby said second lumen (30) is located within said first lumen (20). The first (20) and second (30) lumens are circular in cross section. An elongated hollow tube (40) is connected at a first end (42) to an end of said second lumen (34) that is further from the nostril and connectable at a second end (44) to a carbon dioxide monitoring device (50). The carbon dioxide monitoring device (50) detects the level of carbon dioxide in the portion of the person's exhalation that passes through the second lumen (30). The portion of the person's exhalation that passes through the first lumen (20) is dispersed in the surrounding air. The first lumen (20) also allows inhalation of fresh air by the person. In this embodiment, the second lumen (30) is located at a center position within the circumference of the first lumen (20) so that there is an empty donut shaped space between the first and second lumens. The second lumen (30) is supported by radial supports (63, 64, 65) extending from the inner circumference of the first lumen to the outer circumference of the second lumen. In this embodiment, there are three such radial supports (63, 64, 65) supporting the second lumen (30) and evenly spaced around the circumference. The nasal prong (10) has an enlarged portion and is constructed of flexible material at the end (12) that is inserted into the nostril to provide for easier insertion into nostril of various sizes.

In all the above embodiments, the carbon dioxide monitoring device (50) is of any suitable type. The specific mechanisms of the carbon dioxide monitoring device (50) is not claimed in this specification.

While several particularly preferred embodiments of the present invention have been described and illustrated, it should now be apparent to those skilled in the art that various changes and modifications can be made without departing from the spirit and scope of the invention. Accordingly, the following claims are intended to embrace such changes, modifications, and areas of application that are within the spirit and scope of this invention.

1-12. (canceled)

13. A device to facilitate carbon dioxide sampling in the exhalation breath of a person, comprising:
a single nasal prong insertable into a nostril of the person and allowing gas passage from the nostril into the nasal prong, and the nasal prong having a first lumen, the first lumen allowing gas passage from the nostril to the surrounding external air, and a second lumen, the second lumen located within the first lumen; and
an elongated hollow tube connected at a first end to an end of the second lumen further from the nostril and connectable at a second end to a carbon dioxide monitoring device;
whereby the carbon dioxide monitoring device detects the level of carbon dioxide in the portion of the person's exhalation that passes through the second lumen.

14. The device according to claim 13, wherein the first lumen and the second lumen are cylindrical in shape.

15. The device according to claim 13, wherein the second lumen is located at an off center position within the first lumen such that an outer circumference of the second lumen is in contact with an inner circumference of said first lumen.

16. The device according to claim 13, further comprising a central wall running the length of and dividing the first lumen into two halves, with the second lumen located within one of the halves.

17. The device according to claim 13, wherein the second lumen is located at a center position within the first lumen.

18. The device according to claim 13, wherein the second lumen is supported within the first lumen by a pair of radial supports.

19. The device according to claim 13, wherein the second lumen is supported within the first lumen by three radial supports.

20. The device according to claim 13, further comprising a plurality of radial supports that are spaced out evenly around a circumference of the second lumen.

21. The device according to claim 13, wherein the nasal prong has an enlarged portion at an end that is inserted into the nostril to provide for easier insertion into nostrils of various sizes.

22. The device according to claim 13, wherein the second lumen is the same length as the said first lumen.

23. The device according to claim 13, wherein the second lumen is shorter in length to the first lumen.

24. The device according to claim 13, wherein the second lumen starts from an intermediate position within the first lumen and ends flush with an end of the first lumen that is further from the nostril.
